(19) United States
(12) Patent Application Publication
(10) Pub. No.: US 2025/0014689 A1
(43) Pub. Date: Jan. 9, 2025

(54) METHOD AND SYSTEM FOR DEVELOPING GENERATIVE CHEMISTRY MODEL BASED ON SEQUENTIAL ATTACHMENT-BASED FRAGMENT EMBEDDING (SAFE) REPRESENTATION

(71) Applicant: Quantiphi, Inc., Marlborough, MA (US)

(72) Inventors: Dagnachew Birru, Marlborough, MA (US); Tehemton K Khairabadi, Mumbai (IN); Vishal Pagidipally, Toronto (CA)

(21) Appl. No.: 18/890,958

(22) Filed: Sep. 20, 2024

(51) Int. Cl. G16C 20/50; G06N 3/0455; G06N 3/0475; G06N 3/08; G16C 20/70

(52) U.S. Cl. CPC G16C 20/50; G06N 3/0455; G06N 3/0475; G06N 3/08; G16C 20/70

(57) ABSTRACT

A method and system for developing a generative chemistry model based on SAFE representations is disclosed. The method includes encoding one or more chemical compounds into the SAFE representations. The method may include training an encoder-decoder transformer model based on the SAFE representations using one or more masking techniques. The encoder-decoder transformer model creates a latent space to represent encoded SAFE representations of the chemical compounds. The method may further include training a variational Autoencoder (VAE) to generate a continuous latent space by compressing the latent space of the encoder-decoder transformer model. The method may further include optimizing the continuous latent space to generate a plurality of chemical compounds with specific properties by decoding the SAFE representations of the chemical compounds.

### FIELD OF THE INVENTION

The present disclosure relates to chemical compounds, and more specifically to a method and system for developing a generative chemistry model based on SAFE representations.

### BACKGROUND OF THE INVENTION

The current landscape of machine learning in drug discovery showcases significant advancements in generating compounds within desired property ranges and drug-like scaffolds. State-of-the-art models exhibit a commendable ability to create valid compounds. However, a critical gap remains in the controlled optimization or sampling of these models to produce compounds specific to certain protein targets. This limitation is particularly pronounced when leveraging transformer models, where the discrete nature of the latent space precludes the possibility of performing gradient-based optimization to identify the best binders for protein targets.

Transformer models face inherent challenges in the context of continuous optimization due to their discrete latent spaces. This discretization inhibits the application of gradient-based methods, which are pivotal in fine-tuning and optimizing compounds for specific biological interactions. As a result, the current methodologies fall short in effectively tailoring compounds to enhance binding affinity and selectivity towards designated protein targets.

Moreover, many existing machine learning models are trained on linear representations, such as SMILES (Simplified Molecular Input Line Entry System) or SELFIES (Self-Referencing Embedded Strings). While these representations are useful for generating a broad array of chemical structures, they are not conducive to advanced optimization tasks critical in medicinal chemistry. Specifically, these models struggle with scaffold decoration, linker design, and scaffold morphing processes integral to the iterative optimization cycles in drug design.

Scaffold decoration involves modifying core structures to enhance desired properties, while linker design focuses on connecting functional groups to improve molecular stability and efficacy. Scaffold morphing, another key task, aims to transform molecular frameworks to optimize biological activity and pharmacokinetics. The inability to perform these tasks hampers the transition from initial compound generation to practical drug development, thereby limiting the efficiency and efficacy of the drug discovery pipeline.

Therefore, there is a need for a method and system that may create a continuous latent space for gradient-based optimization.

### SUMMARY

The following embodiments presents a simplified summary in order to provide a basic understanding of some aspects of the disclosed invention. This summary is not an extensive overview, and it is not intended to identify key/critical elements or to delineate the scope thereof. Its sole purpose is to present some concepts in a simplified form as a prelude to the more detailed description that is presented later.

Some example embodiments disclosed herein provide computer-implemented method for developing a generative chemistry model based on SAFE representations, the method may include encoding one or more chemical compounds into the SAFE representations. The method may further include training an encoder-decoder transformer model based on the SAFE representations using one or more masking techniques. The encoder-decoder transformer model creates a latent space to represent encoded SAFE representations of the chemical compounds. The method may further include training a variational Autoencoder (VAE) to generate a continuous latent space by compressing the latent space of the encoder-decoder transformer model. The method may further include optimizing the continuous latent space to generate a plurality of chemical compounds with specific properties by decoding the SAFE representations of the chemical compounds.

According to some example embodiments, wherein the one or more masking technique comprises random masking, substructure span masking, connecting node masking, linker design, random token corruption, substructure corruption, connecting node corruption, random span corruption, scaffold decoration, motif reconstruction, corrupt motif correction, motif extension and sequence to sequence fine tuning.

According to some example embodiments, wherein the encoder-decoder transformer model is a Text-to-Text Transfer Transformer (T5) model.

According to some example embodiments, wherein training the encoder-decoder transformer model comprising generating a plurality of hidden representations for the SAFE representations of chemical compounds.

According to some example embodiments, wherein training the VAE comprising freezing model weights of the encoder-decoder transformer model and recreating the plurality of hidden representations from the continuous latent space.

According to some example embodiments, wherein generating the continuous latent space comprising converting the latent space into a lower dimension continuous latent space for gradient based optimization.

According to some example embodiments, wherein generating the plurality of chemical compounds with specific properties comprising generating a plurality of protein graphical and textual embeddings based on a protein-ligand dataset, mapping the protein graphical and textual embeddings to the continuous latent space, decoding the SAFE representations of the chemical compounds based on the continuous latent space, and validating the plurality of chemical compounds with specific properties using virtual screening and molecular dynamics methods.

According to some example embodiments, wherein validating the plurality of chemical compounds with specific properties comprising masking the SAFE representations of the chemical compounds, determining Tanimoto similarity between an unconditioned generation of chemical compounds and a protein conditioned generation of chemical compounds as ablation study, and comparing with protein decoded SAFE representations and without protein decoded SAFE representations of the chemical compounds.

According to some example embodiments, wherein the optimization of the continuous latent space is gradient-based optimization.

Some example embodiments disclosed herein provide a computer-implemented system for developing a generative chemistry model based on SAFE representations. The computer-implemented system includes a memory, and a processor communicatively coupled the memory, configured to encode one or more chemical compounds into the SAFE representations. The processor further configured to train an encoder-decoder transformer model based on the SAFE representations using one or more masking techniques. The encoder-decoder transformer model creates a latent space to represent encoded SAFE representations of the chemical compounds. The processor further configured to train a variational Autoencoder (VAE) to generate a continuous latent space by compressing the latent space of the encoder-decoder transformer model. The processor further configured to optimize the continuous latent space to generate a plurality of chemical compounds with specific properties by decoding the SAFE representations of the chemical compounds.

Some example embodiments disclosed herein provide a non-transitory computer readable medium having stored thereon computer executable instruction which when executed by one or more processors, cause the one or more processors to carry out operations for developing a generative chemistry model based on SAFE representations, the operations comprising encoding one or more chemical compounds into the SAFE representations. The operations further comprising training an encoder-decoder transformer model based on the SAFE representations using one or more masking techniques. The encoder-decoder transformer model creates a latent space to represent encoded SAFE representations of the chemical compounds. The operations further comprising training a variational Autoencoder (VAE) to generate a continuous latent space by compressing the latent space of the encoder-decoder transformer model. The operations further comprising optimizing the continuous latent space to generate a plurality of chemical compounds with specific properties by decoding the SAFE representations of the chemical compounds.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

The above and still further example embodiments of the present invention will become apparent upon consideration of the following detailed description of embodiments thereof, especially when taken in conjunction with the accompanying drawings, and wherein:

FIG. 1 is a block diagram of an environment of a system of a generative chemistry model based on SAFE representations, in accordance with an example embodiment.

FIG. 2 is a block diagram illustrating various modules within a memory of a computing device configured for updating generative chemistry models for generating chemical compounds with specific properties, in accordance with an example embodiment.

FIG. 3 illustrates a block diagram of architecture of the generative chemistry model, in accordance with an example embodiment.

FIG. 4 illustrates a flow diagram of a method for developing a generative chemistry model based on SAFE representations, in accordance with an example embodiment.

FIG. 5 illustrates a flow diagram of a method for training a variational autoencoder (VAE), in accordance with an example embodiment.

FIG. 6 illustrates a flow diagram of a method for generating the plurality of chemical compounds with specific properties, in accordance with an example embodiment

FIG. 7 illustrates a flow diagram of a method for validating the plurality of chemical compounds with specific properties, in accordance with an example embodiment.

FIG. 8 illustrate an exemplary flow chart for masking the SAFE representations of the chemical compounds, in accordance with an example embodiment.

FIG. 9 illustrates an exemplary User Interface (UI) of masking of the SAFE representations of the chemical compounds, in accordance with an example embodiment.

The figures illustrate embodiments of the invention for purposes of illustration only. One skilled in the art will readily recognize from the following description that alternative embodiments of the structures and methods illustrated herein may be employed without departing from the principles of the invention described herein.

### DETAILED DESCRIPTION

In the following description, for purposes of explanation, numerous specific details are set forth in order to provide a thorough understanding of the present invention. It will be apparent, however, to one skilled in the art that the present invention can be practiced without these specific details. In other instances, systems, apparatuses, and methods are shown in block diagram form only in order to avoid obscuring the present invention.

Reference in this specification to “one embodiment” or “an embodiment” or “example embodiment” means that a particular feature, structure, or characteristic described in connection with the embodiment is included in at least one embodiment of the present invention. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced items. Moreover, various features are described which may be exhibited by some embodiments and not by others. Similarly, various requirements are described which may be requirements for some embodiments but not for other embodiments.

Some embodiments of the present disclosure will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout.

The terms “comprise”, “comprising”, “includes”, or any other variations thereof, are intended to cover a non-exclusive inclusion, such that a setup, device, or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

Furthermore, one or more computer-readable storage media may be utilized in implementing embodiments consistent with the present invention. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., are non-transitory. Examples include random access memory (RAM), read-only memory (ROM), volatile memory, non-volatile memory, hard drives, CD ROMs, DVDs, flash drives, disks, and any other known physical storage media.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present invention. Further, it is to be understood that the phraseology and terminology employed herein are for the purpose of the description and should not be regarded as limiting. Any heading utilized within this description is for convenience only and has no legal or limiting effect.

### Definitions

The term “Structure Aware Fragment Embedding (SAFE) representations may refer to a method of encoding molecular structures by considering their structural fragments and their relationships. SAFE representations aim to capture the chemical and structural context more effectively than traditional representations like SMILES or SELFIES.

The term “generative chemistry model” may refer to a type of machine learning model designed to create novel chemical compounds. These models are trained on existing chemical data to learn patterns and properties of molecules, enabling them to generate new molecular structures that possess desired characteristics, such as specific physical, chemical, or biological properties.

The term “machine learning model” may be used to refer to a computational or statistical or mathematical model that is trained on classical ML modelling techniques with or without classical image processing. The “machine learning model” is trained over a set of data and using an algorithm that it may be used to learn from the dataset.

The term “chemical compound” may refer to a substance composed of two or more different elements that are chemically bonded together in fixed proportions. These elements combine through chemical bonds, such as covalent, ionic, or metallic bonds, to form a molecule or a crystalline structure.

The term “module” used herein may refer to a hardware processor including a Central Processing Unit (CPU), an Application-Specific Integrated Circuit (ASIC), an Application-Specific Instruction-Set Processor (ASIP), a Graphics Processing Unit (GPU), a Physics Processing Unit (PPU), a Digital Signal Processor (DSP), a Field Programmable Gate Array (FPGA), a Programmable Logic Device (PLD), a Controller, a Microcontroller unit, a Processor, a Microprocessor, an ARM, or the like, or any combination thereof.

### End of Definitions

As described earlier, traditional methods of designing curing processes rely heavily on human expertise and conventional numerical methods, which are time-consuming and often suboptimal. The present disclosure addresses these challenges by introducing a method and system for updating prediction model for curing process design. The proposed method and system uses pre-trained and dynamically adaptable Hybrid (physics-informed and data-driven) neural operators coupled with optimizer for effective knowledge and data-driven design generation, testing and evaluation. Further, the proposed method and system iteratively update hybrid neural operators or prediction model with knowledge-driven experiments for curing process design, minimizing human intervention and accelerating the design cycle.

Embodiments of the present disclosure may provide a method, a system, and a computer program product for updating prediction model for curing process design. The method, the system, and the computer program product update prediction model for curing process design in such an improved manner are described with reference to FIG. 1 to FIG. 9 as detailed below.

FIG. 1 illustrates a block diagram of an environment of a system 100 of a generative chemistry model based on Structure Aware Fragment Embedding (SAFE) representations, in accordance with an example embodiment. The system 100 is designed to facilitate efficient and accurate generation of chemical compounds with specific properties by utilizing advanced computational techniques and machine learning models, such as the generative chemistry model. The generative chemistry model may be a merged encoder-decoder transformer model and a Variational autoencoder (VAE) as explained in detail in FIG. 3. The system 100 includes a computing device 102 and an external device 108. The computing device 102 may be communicatively coupled with the external device 108 via a communication network 110. Examples of the computing device 102 may include, but are not limited to, a server, a desktop, a laptop, a notebook, a tablet, a smartphone, a mobile phone, an application server, or the like.

The communication network 110 may be wired, wireless, or any combination of wired and wireless communication networks, such as cellular, Wi-Fi, internet, local area networks, or the like. In one embodiment, the communication network 110 may include one or more networks such as a data network, a wireless network, a telephony network, or any combination thereof. It is contemplated that the data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks, code division multiple access (CDMA), wideband code division multiple access (WCDMA), wireless fidelity (Wi-Fi), wireless LAN (WLAN), Bluetooth®, Internet Protocol (IP) data casting, satellite, mobile ad-hoc network (MANET), and the like, or any combination thereof.

The computing device 102 may include a memory 104, and a processor 106. The term “memory” used herein may refer to any computer-readable storage medium, for example, volatile memory, random access memory (RAM), non-volatile memory, read only memory (ROM), or flash memory. The memory 104 may include a Random-Access Memory (RAM), a Read-Only Memory (ROM), a Complementary Metal Oxide Semiconductor Memory (CMOS), a magnetic surface memory, a Hard Disk Drive (HDD), a floppy disk, a magnetic tape, a disc (CD-ROM, DVD-ROM, etc.), a USB Flash Drive (UFD), or the like, or any combination thereof.

The term “processor” used herein may refer to a hardware processor including a Central Processing Unit (CPU), an Application-Specific Integrated Circuit (ASIC), an Application-Specific Instruction-Set Processor (ASIP), a Graphics Processing Unit (GPU), a Physics Processing Unit (PPU), a Digital Signal Processor (DSP), a Field Programmable Gate Array (FPGA), a Programmable Logic Device (PLD), a Controller, a Microcontroller unit, a Processor, a Microprocessor, an ARM, or the like, or any combination thereof.

The processor 106 may retrieve computer program code instructions that may be stored in the memory 104 for execution of the computer program code instructions. The processor 106 may be embodied in a number of different ways. For example, the processor 106 may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor 106 may include one or more processing cores configured to perform independently. A multi-core processor may enable multiprocessing within a single physical package. Additionally, or alternatively, the processor 106 may include one or more processors configured in tandem via the bus to enable independent execution of instructions, pipelining, and/or multithreading.

Additionally, or alternatively, the processor 106 may include one or more processors capable of processing large volumes of workloads and operations to provide support for big data analysis. In an example embodiment, the processor 106 may be in communication with a memory 104 via a bus for passing information among components of the system 100.

The memory 104 may be non-transitory and may include, for example, one or more volatile and/or non-volatile memories. In other words, for example, the memory 104 may be an electronic storage device (for example, a computer readable storage medium) comprising gates configured to store data (for example, bits) that may be retrievable by a machine (for example, a computing device like the processor 106). The memory 104 may be configured to store information, data, contents, applications, instructions, or the like, for enabling the apparatus to carry out various functions in accordance with an example embodiment of the present disclosure. For example, the memory 104 may be configured to buffer input data for processing by the processor 106.

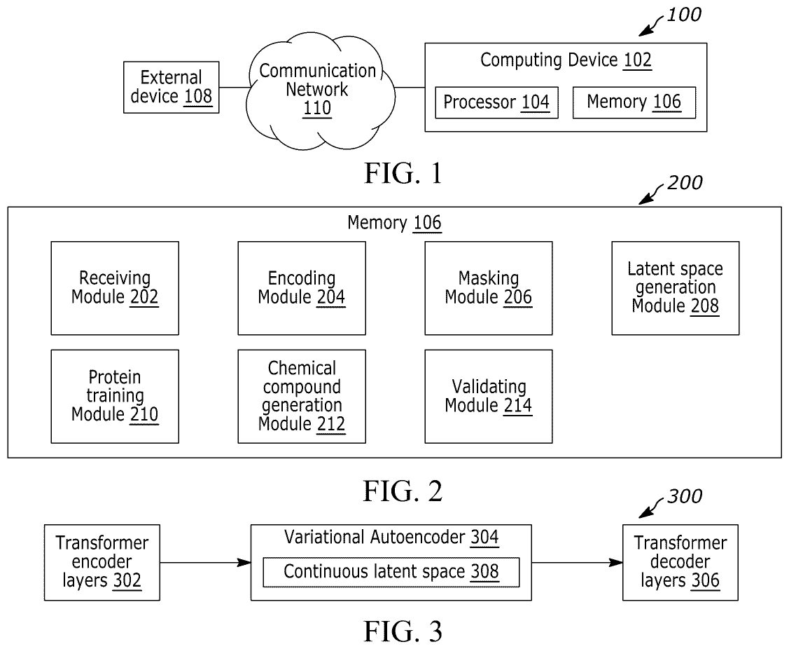

The computing device 102 may be capable of updating the generative chemistry model for generating chemical compounds with specific properties. The memory 104 may store instructions that, when executed by the processor 106, cause the computing device 102 to perform one or more operations of the present disclosure which will be described in greater detail in conjunction with FIG. 2. The computing device 102 is responsible for performing tasks such as encoding chemical compounds into SAFE representations, training a transformer model, training a variational autoencoder (VAE), and optimizing continuous latent space. The computing device 102 continually learns and updates the generative chemistry model based on SAFE representation of chemical compounds, ensuring that the optimization component iteratively improves the generation of chemical compounds with specific properties using feedback from both simulations and real-world experiments.

The external devices 108 may refers to various hardware and software tools that may be integrated with the system 100 to enhance its functionality. These devices may include database of SAFE representation of chemical compounds. This data is essential for the continual learning framework to make accurate updates to the generative chemistry model and ensure the optimization component may refine the latent space of the generative chemistry model. The complete process followed by the system 100 is explained in detail in conjunction with FIG. 2 to FIG. 9.

FIG. 2 illustrates a block diagram 200 illustrating various modules within the memory 104 of the computing device 102 configured for updating generative chemistry models for generating chemical compounds with specific properties, in accordance with an example embodiment. The memory 104 may include a receiving module 202, an encoding module 204, a masking model 206, a latent space generation module 208, a protein mapping module 210, a chemical compound generation module 212, and a validating module 214.

The receiving module 202 is responsible for receiving input data corresponding to the generative chemistry model. The input data may include chemical formulae of a plurality of chemical compounds, database of chemical compounds, Simplified Molecular Input Line Entry System (SMILES) representation, Selfies representation, Sequential Attachment-Based Fragment Embedding (SAFE) representations of the chemical compounds, binders, protein-ligand database, etc. The input data may be used for training the generative chemistry model to generate chemical compounds with specific properties. The receiving module 202 ensures that the computing device 102 has the latest and most accurate input data necessary to update the prediction model. Further, the receiving module 202 may store the input data in the memory 106 for optimizing and re-training of the generative chemistry model.

The encoding module 204 is configured to encode the chemical formulae of plurality of chemical compounds, database of chemical compounds, SMILES representations, Selfies representation into the SAFE representations of chemical compounds. The encoding module 204 utilizes the input data received from the receiving module 202 to homogenise the input data for training of the generative chemistry model. In an embodiment, the encoding module 204 may encode SMILES strings as an unordered sequence of interconnected fragment blocks while maintaining compatibility with existing SMILES parsers. SAFE representations streamline complex generative tasks, including scaffold decoration, fragment linking, polymer generation, and scaffold hopping. The generative tasks are performed by the generative chemistry model to generate chemical compounds with specific properties, such as a chemical compound capable of binding with specific proteins.

The masking model 206 is responsible for masking one or more parts of the SAFE representations of the chemical compounds before training the generative chemistry model. The masking module 206 may implement a plurality of masking techniques to optimize the generative chemistry model. The plurality of masking techniques implemented by the masking module 206 may include random masking, scaffold decoration, linker design, motif reconstruction or corrupted motif correction and motif extension. Further, the masked SAFE representations are used to train the encoder-decoder transformer model of the generative chemistry model. The encoder-decoder transformer model may represent the SAFE representations in its latent space. In some embodiments, the encoder-decoder transformer model is optimized by backpropagating the training loss through the latent space of the encoder-decoder transformer model.

Upon training the encoder-decoder transformer model, the latent space generation module 208 is configured to generate a continuous latent space for the generative chemistry model. The continuous latent space is generated by compressing the latent space of the encoder-decoder transformer model through the VAE. The continuous latent space retains the properties of the latent space of the encoder-decoder transformer model, such as the SAFE representations of the chemical compounds. The latent space generation module 208 freezes the weight of the encoder-decoder transformer model. The trained encoder-decoder transformer model may be used to generate hidden representations for the SAFE representations and the VAE is trained to recreate those hidden states from the latent space. This joint training paradigm ensure that the continuous latent space of the generative chemistry is shaped correctly and is compatible with downstream property optimization tasks, such as generating chemical compounds with specific properties.

After creating the continuous latent space, the protein training module 210 may train the generative chemistry model on a plurality of protein embeddings. The plurality of protein embeddings may be retrieved from the protein-ligand database. The protein-ligand database may be encoded into the SAFE representations by the encoding module 204 to train the generative chemistry model. The protein training module 210 may map the protein embedding to decoding layers of the generative chemistry model. It should be noted that the protein mapping module 210 may map the proteins which are target proteins, such as the proteins intended to be bind with the chemical compounds generated by the generative chemistry model.

The chemical compound generation module 212 is configured to generate the chemical compounds with specific properties by the generative chemistry model. The chemical compound generation module 212 may decode the SAFE representations of the chemical compounds by decoding layers of the generative chemistry model. The decoding also include mapping the decoded SAFE representations with the protein embeddings mapped in the decoding layers. Further, to generate the chemical compounds with specific properties, the chemical compound generation module 212 may perform gradient based optimization on the continuous latent space optimization to find binder scaffolds with good, predicted binding affinity.

The validating module 214 is responsible for validating the generated chemical compounds by the chemical compound generation module 212. The validation module 214 masks input chemical compounds binding motifs and checks the Tanimoto similarity between the unconditioned generation and protein conditioned generation as ablation studies. Further, the latent vector of the generative chemistry model is heavily noised and the decoded compounds with and without protein embedding conditioning are compared with each other.

FIG. 3 illustrates a block diagram of architecture of the generative chemistry model 300, in accordance with an example embodiment. The generative chemistry model 300 include the encoder-decoder transformer model and the Variational autoencoder (VAE). The encoder-decoder transformer model is the T5 transformer model configured to process the SAFE representation of the chemical compounds. The encoder-decoder transformer model may include a plurality of transformer encoder layers 302, a laten space and a plurality of transformer decoder layers 304. The plurality of transformer encoder layers 302 is configured to take the SAFE representation of the chemical compounds for training. The SAFE representation of the chemical compounds is in alphanumeric format. The plurality of transformer encoder layers 302 is neural network layers configured to extract features of the SAFE representation of the chemical compounds. Further, the extracted features of the SAFE representation of the chemical compounds are represented on the latent space of the encoder-decoder transformer model. The latent space may be a discrete latent space.

Further, the VAE 304 is placed between the plurality of transformer encoder layers 302 and the plurality of transformer decoder layers 306. The VAE 304 of the generative chemistry model 300 is configured to compress the latent space to generate a continuous latent space 308 for the generative chemistry model. The VAE 304 may also include a plurality of encoder layers and a plurality of decoder layers. The transformer decoder layers 306 is configured to decode the extracted features of the SAFE representations of the chemical compounds. The transformer decoder layers 306 may include mapping of the proteins embeddings. The transformer decoder layers 306 maps the protein embeddings with the SAFE representations of the chemical compounds to generate the chemical compounds with specific properties as explained in further in detail. By fine tuning the generative chemistry model 300 using merged graphical and textual embeddings of the target proteins as the keys for the decoder may enable the model to directly generate the relevant drug-like compounds with very high predicted binding affinity. This may also speed up the gradient based optimization process.

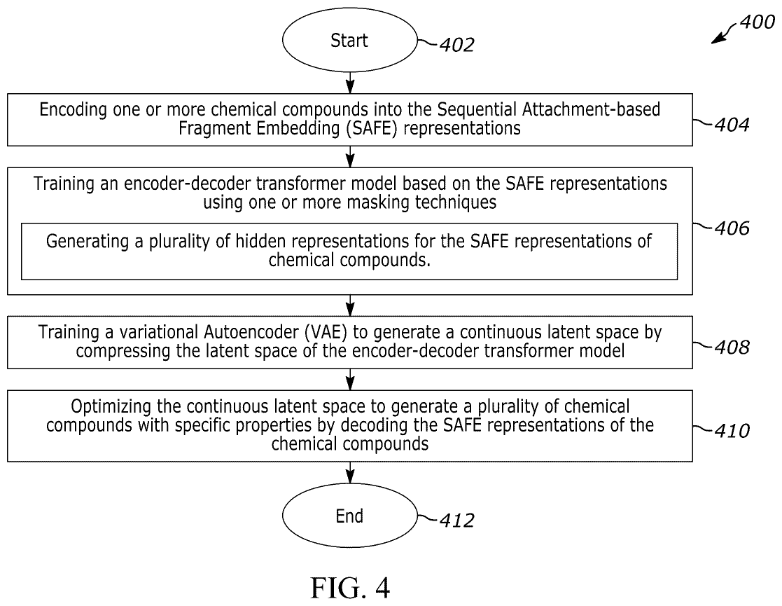

FIG. 4 illustrates a flow diagram of a method 400 for developing a generative chemistry model based on SAFE representations, in accordance with an example embodiment. It will be understood that each block of the flow diagram of the method 400 may be implemented by various means, such as hardware, firmware, processor, circuitry, and/or other communication devices associated with execution of software including one or more computer program instructions. For example, one or more of the procedures described above may be embodied by computer program instructions. In this regard, the computer program instructions which embody the procedures described above may be stored by a memory 104 of the computing device 102, employing an embodiment of the present disclosure and executed by a processor 106. As will be appreciated, any such computer program instructions may be loaded onto a computer or other programmable apparatus (for example, hardware) to produce a machine, such that the resulting computer or other programmable apparatus implements the functions specified in the flow diagram blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flow diagram blocks.

Accordingly, blocks of the flow diagram support combinations of means for performing the specified functions and combinations of operations for performing the specified functions for performing the specified functions. It will also be understood that one or more blocks of the flow diagram, and combinations of blocks in the flow diagram, may be implemented by special purpose hardware-based computer systems which perform the specified functions, or combinations of special purpose hardware and computer instructions.

The method 400 illustrated by the flow diagram of FIG. 4 for developing a generative chemistry model based on SAFE representations may start at step 402. Further, the method 400 may include encoding one or more chemical compounds into SAFE representations, at step 404. The one or more chemical compounds may be received in the form of chemical formulas, SMILES representations, and Selfies representations retrieved from database, protein-ligand database, etc. The chemical compounds are encoded as SAFE representations to train the generative chemistry model for generating chemical compounds with specific properties. The SAFE representations of the chemical compounds are represented in alphanumeric form, one example of a SAFE representation of chemical compound is N18CCCCC1.0=C6C#CC8.N67.c17ccc2ncnc4c2c1.N45.c15cccc(Br)c1 corresponding to a SMILES representation O=C(C#CCNICCCCC1)Nc1ccc2ncnc(Nc3cccc(Br)c3)c2c1. Further, the SAFE representations may streamline complex generative tasks, including scaffold decoration, fragment linking, polymer generation, and scaffold hopping to be performed at later steps.

The method 400, at step 406, may include training an encoder-decoder transformer model based on the SAFE representations using one or more masking techniques. The encoder-decoder transformer model creates a latent space to represent encoded SAFE representations of the chemical compounds. Further, training the encoder-decoder transformer model includes generating a plurality of hidden representations for the SAFE representations of chemical compounds. The encoder-decoder transformer model is a Text-to-Text Transfer Transformer (T5) model to train based on the SAFE representation of the chemical compounds. Further, training the encoder-decoder transformer model based on the SAFE representations may include masking one or more parts of the SAFE representations of the chemical compounds. The encoder-decoder transformer model may include a plurality of masking techniques to mask a part of the SAFE representations of the chemical compounds to generate new chemical compounds with specific properties. The plurality of masking techniques may include random masking, scaffold decoration, linker design, motif reconstruction or corrupted motif correction and motif extension. The masking of SAFE representations is important to train and generate new chemical compounds as the masked part is generated based on the desired properties of the intended chemical compounds.

At step 408, the method 400 may include, training a variational Autoencoder (VAE) 304 to generate a continuous latent space 308 by compressing the latent space of the encoder-decoder transformer model. The latent space is compressed to a lower dimension to generate the continuous latent space 308 for gradient based optimization. It should be noted that the continuous latent space 308 retains properties of the latent space of the encoder-decoder transformer model. Further, the VAE 304 decodes the hidden representations generated by the encoder-decoder transformer model as the models are jointly trained. This joint training paradigm ensure that the continuous latent space 308 of the generative chemistry is shaped correctly and is compatible with downstream property optimization tasks, such as generating chemical compounds with specific properties. Further, the continuous latent space 308 of the VAE 304 may be embedded with the protein embeddings to generate the chemical compounds with binding affinity to specific proteins. The protein embeddings may be in textual or graphical form corresponding to the keys mapped in the decoding layers of the VAE 304.

At step 410, the method 400 may include, optimizing the continuous latent space 308 to generate a plurality of chemical compounds with specific properties by decoding the SAFE representations of the chemical compounds. This step is critical for generating chemical compounds with desired and specific properties. Further, generation of new chemical compounds includes mapping the protein embeddings with the masked SAFE representations of the chemical compounds in the continuous latent space 308 of the generative chemistry model. The optimization of the continuous latent space 308 is gradient-based optimization. The gradient based optimization may perform scaffold decoration, linker design, and scaffold morphing to generate and validate the generated chemical compounds with specific properties. Further, the method 400 terminated at step 412.

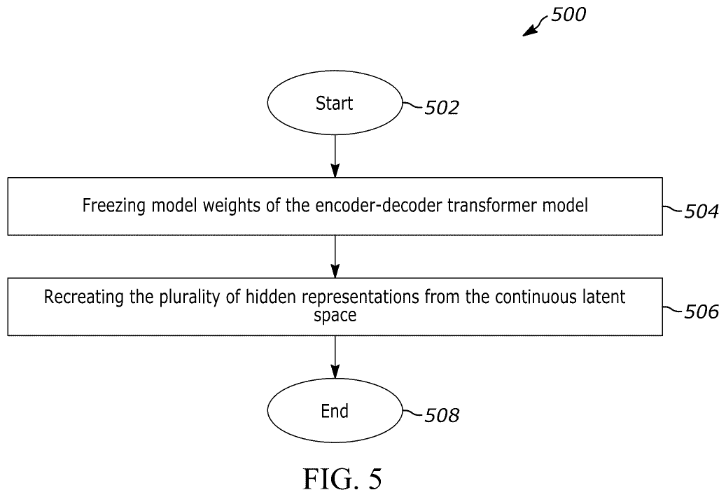

FIG. 5 illustrates a flow diagram of a method 500 for training a variational autoencoder (VAE) 304, in accordance with an example embodiment. It will be understood that each block of the flow diagram of the method 500 may be implemented by various means, such as hardware, firmware, processor, circuitry, and/or other communication devices associated with execution of software including one or more computer program instructions. For example, one or more of the procedures described above may be embodied by computer program instructions. In this regard, the computer program instructions which embody the procedures described above may be stored by a memory 104 of the computing device 102, employing an embodiment of the present disclosure and executed by a processor 106. As will be appreciated, any such computer program instructions may be loaded onto a computer or other programmable apparatus (for example, hardware) to produce a machine, such that the resulting computer or other programmable apparatus implements the functions specified in the flow diagram blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flow diagram blocks.

Accordingly, blocks of the flow diagram support combinations of means for performing the specified functions and combinations of operations for performing the specified functions for performing the specified functions. It will also be understood that one or more blocks of the flow diagram, and combinations of blocks in the flow diagram, may be implemented by special purpose hardware-based computer systems which perform the specified functions, or combinations of special purpose hardware and computer instructions.

FIG. 5 is explained in conjunction with elements from FIGS. 1, 2, 3, and 4. At step 502, the method 500 is initiated. The method 500, at step 504 may include freezing the model weights of the encoder-decoder transformer model. The model weights are a subset of the parameters in a model that represent the strength of connections between variables. During training, the model adjusts these model weights to optimize its performance. Model weights may determine how input tokens are transformed as they pass through the layers of the model. Further, the input tokens here refer to the SAFE representations of the chemical compounds used in training the encoder-decoder transformer model. By freezing the model weights of the encoder-decoder transformer model, the VAE 304 is being trained on above the encoder-decoder transformer model compressing the latent space to generate a continuous latent space 308.

Further, the method 500, at step 506, may include recreating the plurality of hidden representations from the continuous latent space 308. The hidden representations are decoded by the decoding layers of the VAE 304. The method 500 ends at step 508.

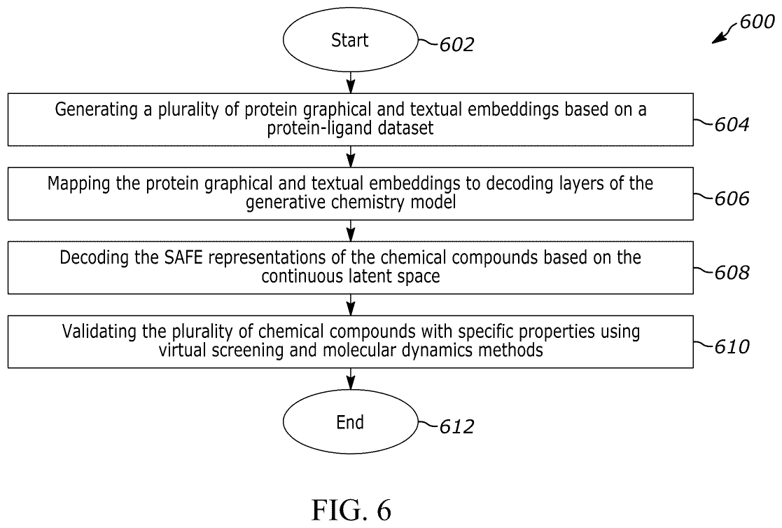

FIG. 6 illustrates a flow diagram of a method 600 for generating the plurality of chemical compounds with specific properties, in accordance with an example embodiment. It will be understood that each block of the flow diagram of the method 600 may be implemented by various means, such as hardware, firmware, processor, circuitry, and/or other communication devices associated with execution of software including one or more computer program instructions. For example, one or more of the procedures described above may be embodied by computer program instructions. In this regard, the computer program instructions which embody the procedures described above may be stored by a memory 104 of the computing device 102, employing an embodiment of the present disclosure and executed by a processor 106. As will be appreciated, any such computer program instructions may be loaded onto a computer or other programmable apparatus (for example, hardware) to produce a machine, such that the resulting computer or other programmable apparatus implements the functions specified in the flow diagram blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flow diagram blocks.

Accordingly, blocks of the flow diagram support combinations of means for performing the specified functions and combinations of operations for performing the specified functions for performing the specified functions. It will also be understood that one or more blocks of the flow diagram, and combinations of blocks in the flow diagram, may be implemented by special purpose hardware-based computer systems which perform the specified functions, or combinations of special purpose hardware and computer instructions.

FIG. 6 is explained in conjunction with elements from FIGS. 1, 2, 3, 4, and 5. The method 600 is started at step 602. At step 604, the method 600 may include generating a plurality of protein graphical and textual embeddings based on a protein-ligand dataset. In an embodiment, datasets such as binding database may be utilized where known binders to proteins are expressed along with other information, such as their binding affinity, association constants, dissociation constants etc. The protein-ligand pairs that are known to have good binding affinity are identified, after applying threshold based filters over the binding affinity scores. The protein chains that contain the binding pocket are then picked from the database, or the entire protein sequence may be picked if some other database where the pocket sequence is not given.

Further, the protein sequence is then passed through protein language models, such as ANKH, ESM, ProtBert, etc. to generate the protein embeddings. Subsequently, PDB structures of these proteins may be used to encode into a graphical structure and compute the graphical structural embeddings of the protein. The protein embeddings may be projected to the decoder cross attention layer sizes using a simple feed forward layer acting as a projection matrix. This step is done to ensure that all the layers have the correct shape. Further, the projected embedding space may be used as cross attention in the transformer decoder layer for decoding chemical compounds conditioned to the protein.

The method 600, at step 606 may further include mapping the protein graphical and textual embeddings to decoding layers of the generative chemistry model as the keys may enable the generative chemistry model to map the areas of the continuous latent space 308 to the protein landscape.

Further, the method 600, at step 608 may further include decoding the SAFE representations of the chemical compounds based on the continuous latent space 308 and the protein embeddings. The decoding layers of the generative chemistry model may decode the representations of the chemical compounds from the continuous latent space 308. In an embodiment, the decoding layers may map the SAFE representations with each of the protein embeddings to generate the chemical compounds which have good binding affinity with the specific proteins. In an embodiment, during fine tuning the generative chemistry model, several hundred thousand pairs may be used to fine tune the model such that it learns the relationship between the protein and chemical representations and may decode compounds that are in theory expected to have higher specificity and binding affinity towards the protein target.

Further, the method 600, at step 610 may further include validating the plurality of chemical compounds with specific properties using virtual screening and molecular dynamics methods. The virtual screening of the chemical compounds may refer to the application of computational techniques to the selection compounds for biological screening, either from in-house databases, externally available compound collections, or from virtual libraries, that is, sets of compounds that could potentially be synthesized. Further, the Molecular dynamics (MD) is a computer simulation method for analysing the physical movements of atoms and molecules. The atoms and molecules are allowed to interact for a fixed period of time, giving a view of the dynamic “evolution” of the system. Examples of molecular dynamics may include charge-charge interactions between ions and dipole-dipole interactions between molecules. The method 600 terminates at step 612.

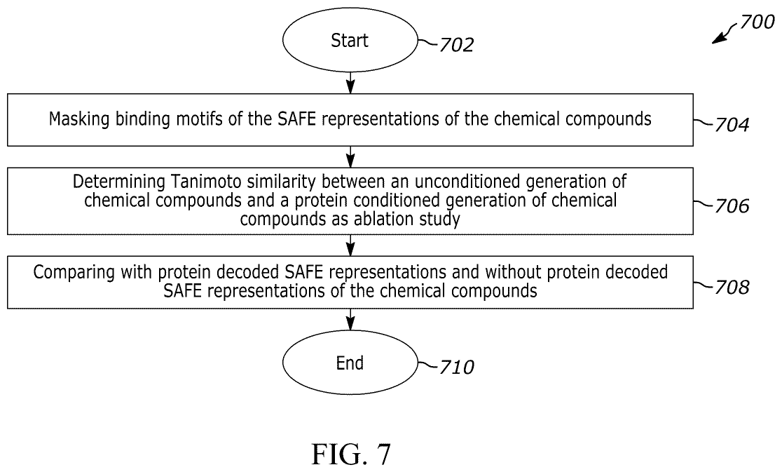

FIG. 7 illustrates a flow diagram of a method 700 for validating the plurality of chemical compounds with specific properties, in accordance with an example embodiment. It will be understood that each block of the flow diagram of the method 700 may be implemented by various means, such as hardware, firmware, processor, circuitry, and/or other communication devices associated with execution of software including one or more computer program instructions. For example, one or more of the procedures described above may be embodied by computer program instructions. In this regard, the computer program instructions which embody the procedures described above may be stored by a memory 104 of the computing device 102, employing an embodiment of the present disclosure and executed by a processor 106. As will be appreciated, any such computer program instructions may be loaded onto a computer or other programmable apparatus (for example, hardware) to produce a machine, such that the resulting computer or other programmable apparatus implements the functions specified in the flow diagram blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flow diagram blocks.

Accordingly, blocks of the flow diagram support combinations of means for performing the specified functions and combinations of operations for performing the specified functions for performing the specified functions. It will also be understood that one or more blocks of the flow diagram, and combinations of blocks in the flow diagram, may be implemented by special purpose hardware-based computer systems which perform the specified functions, or combinations of special purpose hardware and computer instructions.

FIG. 7 is explained in conjunction with elements from FIGS. 1, 2, 3, 4, 5, and 6. At step 702, the method 700 is initiated. The method 700, at step 704 may include masking binding motifs of the SAFE representations of the chemical compounds.

Further, the method 700, at step 706, may include determining a similarity between an unconditioned generation of chemical compounds and a protein conditioned generation of chemical compounds as ablation study. The similarity may be determined via, a matching algorithm, a Dice similarity, a Cosine similarity, a Sokal index, a Russel-Rao index, a Kluczynski index, a McConnaughey coefficient, a Tversky index. Besides fingerprint similarity, 2D and 3D structural similarity methods may also be used. The ablation study may be used to investigate the performance of the generative chemistry model by removing certain components to understand the contribution of the component to the overall model.

In an embodiment, the ablation study protocol may start with N number of random noise latent vectors decoded into valid compounds. The compounds which are generated using protein embeddings may then be attached to the target protein and their average binding affinities is calculated.

Further, the method 700, at step 708, further include comparing with protein decoded SAFE representations and without protein decoded SAFE representations of the chemical compounds. This step is critical to check the efficiency and optimization of the generative chemistry model. Further, to optimize the generative chemistry model cross-attention weights of the encoder-decoder transformer model and the generative chemistry model are interpreted. The method 700 ends at step 710.

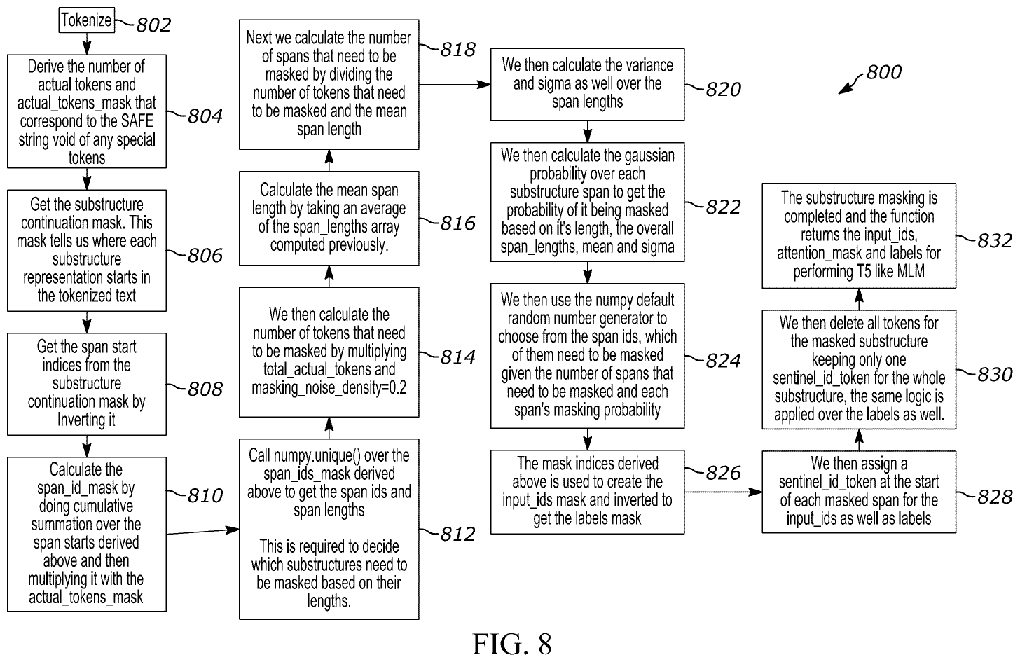

FIG. 8 illustrates a flow diagram of a method 800 for generating a second set of experiments when the curing coefficients are unavailable, in accordance with an example embodiment. It will be understood that each block of the flow diagram of the method 800 may be implemented by various means, such as hardware, firmware, processor, circuitry, and/or other communication devices associated with execution of software including one or more computer program instructions. For example, one or more of the procedures described above may be embodied by computer program instructions. In this regard, the computer program instructions which embody the procedures described above may be stored by a memory 104 of the computing device 102, employing an embodiment of the present disclosure and executed by a processor 106. As will be appreciated, any such computer program instructions may be loaded onto a computer or other programmable apparatus (for example, hardware) to produce a machine, such that the resulting computer or other programmable apparatus implements the functions specified in the flow diagram blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flow diagram blocks.

Accordingly, blocks of the flow diagram support combinations of means for performing the specified functions and combinations of operations for performing the specified functions for performing the specified functions. It will also be understood that one or more blocks of the flow diagram, and combinations of blocks in the flow diagram, may be implemented by special purpose hardware-based computer systems which perform the specified functions, or combinations of special purpose hardware and computer instructions.

FIG. 8 illustrate an exemplary flow chart 800 for masking the SAFE representations of the chemical compounds, in accordance with an example embodiment. Initially, at step 802, the SAFE representations of the chemical compounds are tokenized. Tokenization is the process of replacing sensitive data with unique identification symbols that retain all the essential information about the data without compromising its security. The SAFE representations are tokenized in a way that essential molecules of the chemical compounds are tokenized retaining the properties of the chemical compound. Thus, the tokenization process creates a tokenized version of the chemical compound consisting small tokens of essential molecules connected with each other.

At step 804, the number of actual tokens and actual_tokens_mask are derived that correspond to the SAFE representation of the chemical compounds void of any special tokens. In some embodiments, the actual_tokens_mask may be a binary tensor mask that may look like [0111101100000]. The binary tensor mask may indicate the locations of actual tokens and masks out (0) positions that correspond to special tokens including and not limited to pad, mask, sentinel_tokens, cos, bos, sep, etc.

At step 806, get the substructure continuation mask. The substructure continuation mask determines where each substructure representation starts in the tokenized text. In simpler words, the substructure continuation mask is used to indicate the start of each substructure within the tokenized representation of the chemical compound. The substructure continuation mask helps in identifying where each substructure begins, which is crucial for tasks like substructure-based modelling and optimization. The substructure continuation mask marks the tokens that correspond to continuation of a fragment, such as continuations as 1 and fragment starts as 0.

At step 808, get the span start indices from the substructure continuation mask by inverting it. The substructure continuation mask marks the tokens that correspond to continuation of a fragment, such as continuations as 1 and fragment starts as 0. The substructure continuation mask effectively highlights the positions in the sequence that correspond to the start of substructures. By identifying substructure continuations and then inverting the mask that has 1 wherever a new substructure span of tokens begins. The process effectively highlights the positions in the sequence that are not the start of substructures. Further, span start indices are the positions in the original token sequence of the chemical compound where the inverted mask has 1. These positions mark where substructures are not starting, essentially highlighting the spans of the substructures.

At step 810, calculate the span_ids_mask by doing cumulative summation over the span starts mask. The span start mask may be first bitwise ANDed with the inverted special_tokens_mask to get the positions that relate to the start of actual substructure spans without any special tokens. The result is then cumulatively summed over to get the span_ids_mask. The mask helps to uniquely identify the spans of each substructure within the tokenized representation of the chemical compound.

At step 812, call NumPy unique( ) over the span_ids_mask derived above to get the span ids and span lengths. This may be required to decide which substructures need to be masked based on their lengths. By using NumPy's unique( ) function, the unique span IDs and their corresponding lengths from the span_ids_mask are identified. This information is crucial for deciding which substructures need to be masked based on their lengths, facilitating more efficient and targeted substructure analysis and optimization tasks.

At step 814, calculate the number of tokens that need to be masked by multiplying total actual_tokens and masking_noise_density. The masking_noise_density may be any value between 0.05-0.45 realistically depending on the generative chemistry model. By calculating the total number of actual tokens and multiplying by the masking noise density (0.2), the number of tokens is determined that need to be masked. By way of an example, a total of 5 actual tokens and a masking noise density of 0.2, the result is that 1 token needs to be masked.

At step 816, the mean span length is calculated by taking an average of the span_lengths array computed previously. By taking the average of the span_lengths array, the mean span length is calculated.

At step 818, the number of spans is calculated that need to be masked by dividing the number of tokens that need to be masked and the mean span length. By dividing the number of tokens that need to be masked by the mean span length and rounding up, the number of spans is determined that need to be masked. By way of an example, with 1 token to mask and a mean span length of approximately 2.67, the result is that 1 span needs to be masked.

At step 820, the variance and sigma as well over the span lengths are calculated. By calculating the variance and standard deviation (sigma) of the span lengths, measure of the dispersion or variability in the span lengths.

At step 822, the gaussian probability is calculated over each substructure span to get the probability of it being masked based on its length, the overall span_lengths, mean and sigma. Further, calculating the gaussian probability may include calculating the mean and standard deviation and computing the gaussian probability. The Gaussian probability provides the likelihood of each span length being masked based on the distribution of span lengths. By way of an example, for span lengths [4, 2, 2], the computed probabilities may reflect how likely each span length is to be selected for masking, with those closer to the mean having higher probabilities.

At step 824, the NumPy default random number generator is used to choose from the span ids, which of them need to be masked given the number of spans that need to be masked and each span's masking probability.

At step 826, The mask indices derived above is used to create the input_ids mask and inverted to get the labels mask. The input_ids mask refers to a mask indicating which tokens (or spans) are selected for masking. It may have 1 at the positions that are masked and 0 elsewhere. The labels mask may refer to the inverted mask of the input_ids mask. It may have 1 at the positions that are not masked (i.e., original tokens) and 0 at the masked positions.

At step 828, a sentinel_id_token is assigned at the start of each masked span for the input_ids as well as labels. The sentinel_id_token may refer to a special token used to indicate the start of a masked span. It should be noted that sentinel_id_token is unique and not used in normal sequences. By assigning the sentinel_id_token at the start of each masked span, the beginning of each masked span is marked in both input_ids and labels. This helps in identifying and processing masked spans efficiently in model training and evaluation.

At step 830, all tokens for the masked substructure are deleted keeping only one sentinel_id_token for the whole substructure, the same logic is applied over the labels as well. By replacing all tokens within each masked substructure with a sentinel token ID, the masking process is simplified for both input_ids and labels. This approach ensures that the entire substructure is consistently marked as masked, with only the sentinel token ID remaining.

Finally at step 832, the substructure masking is completed, and the function returns the input_ids, attention_mask and labels for training encoder-decoder transformer model. The function returns the input_ids, attention_mask, and labels for encoder-decoder transformer model. The input_ids and labels include the sentinel_id_token for masked substructures, while the attention_mask ensures that only relevant tokens are attended to during model training.

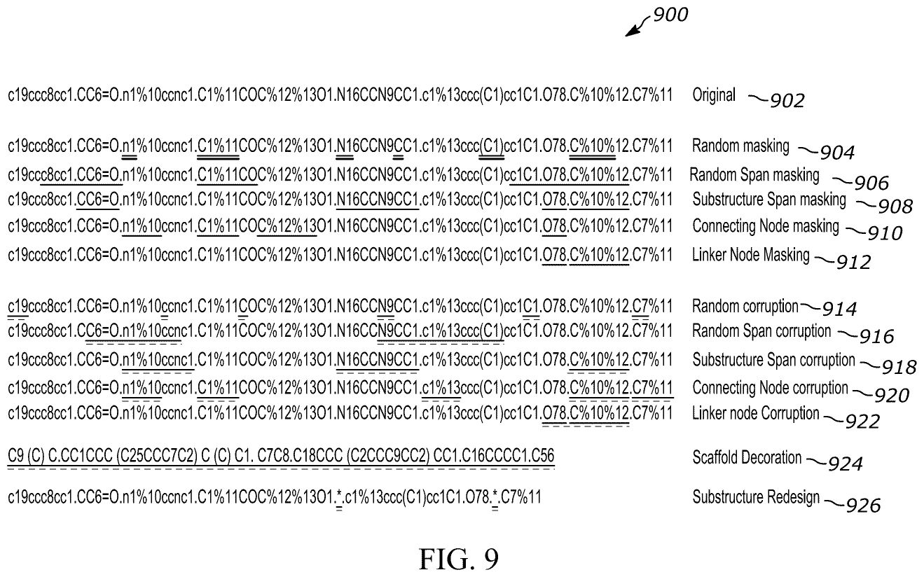

FIG. 9 illustrates an exemplary User Interface (UI) 900 of masking of the SAFE representations of the chemical compounds, in accordance with an example embodiment. The UI 900 may be implemented on the external device 108. An original SAFE representation 902 of a chemical compound is illustrated. The original SAFE representation 902 may be masked by using a plurality of masking strategies. The plurality of masking strategies may include masking, corruption, decoration, and seq-to-seq fine tuning.

The masking strategy may include a <gen> sentinel token for generation mode switching. The <gen> sentinel token acts as a mode-switching mechanism, guiding the model to focus on generation tasks when encountered. It is particularly useful in multi-task models where different types of processing or tasks are required. This approach ensures that the model understands when it should generate new data versus performing other tasks, like detecting corruptions or reconstructing sequences. Further, the masking strategy include a random masking strategy which may randomly mask 15% of the tokens of the original SAFE representations 902 of the chemical compound. The random masking strategy may generate the masked SAFE representations 904 as illustrated in FIG. 9.

The masking strategy include a Random span masking strategy that may mask out a random span. The Random span masking strategy may generate the masked SAFE representations 906. Further, the masking strategy include a Substructure span masking strategy that may identify a substructure from the SAFE representation and mask it out. The Substructure span masking strategy may generate the masked SAFE representations 908.

Further, the masking strategy include connecting node masking strategy that may mask out the atoms that are involved in the substructures interacting. This helps the model understand how these substructures are interacting with each other. The connecting node masking strategy may generate the masked SAFE representations 910. The masking strategy may further include Linker node masking strategy that may mask the linker span between 2 substructures. The Linker node masking strategy may generate the masked SAFE representations 912.

The corruption strategy may include a <rec> sentinel token for corrupted smiles reconstruction. This make the generative chemistry model converge much faster by performing corrupt token detection and original text retrieval. Moreover, this may also train the decoder to be more robust towards generating invalid smiles fragments. The corruption strategy may include random token corruption that may randomly replace some tokens with others and ask the model to perform Replaced Token Detection. The random token corruption may generate the masked SAFE representations 914.

Further, the corruption strategy may include Random span corruption strategy that may randomly corrupt a span of tokens and ask the model to recover the original sequence. The Random span corruption strategy may generate the masked SAFE representations 916. Further, the corruption strategy may include Substructure corruption strategy that may pick a substructure from the SAFE representation 902 and replace it with random tokens and ask the model to recover the original sequence. The substructure corruption strategy may generate the masked SAFE representations 918. The corruption strategy may include Connecting node corruption strategy that may replace the interacting atoms with either sentinel token like in encoder-decoder transformer model or random tokens. The connecting node corruption strategy may generate the masked SAFE representations 920.

Further, the decoration strategy may include a <dec> sentinel token for scaffold decoration. The decoration strategy may generate the scaffold smiles and reconstruct the original compound using it. This may be considered as an extreme case of span corruption. The most extreme case may be to generate the entire compound scaffold using MakeScaffoldGeneric( ) from rdkit. The decoration strategy may include substructure scaffold decoration using MurckoScaffold.GetScaffoldForMol( ) and a complete scaffold decoration using MurckoScaffold.GetScaffoldForMol( ) The scaffold decoration strategy may generate the masked SAFE representations 924. It should be noted that the decoration strategy is done a seq-to-seq fine tuning.

Further, a substructure redesign strategy may replace the entire substructure with an * symbol. This is similar to span corruption or span masking but may help better align the model to downstream redesign use cases where replace a substructure with an * to sample. The substructure redesign strategy may generate the masked SAFE representations 926.

As will be appreciated by those skilled in the art, the techniques described in the various embodiments discussed above are not routine, or conventional, or well understood in the art. The techniques discussed above provide for innovative solutions to address the challenges associated with developing the generative chemistry model for generating chemical compounds with specific properties. The disclosed techniques offer several advantages over the existing methods:

Improved Compound Optimization: The continuous latent space created by the model facilitates gradient-based optimization, allowing for more precise tailoring of chemical compounds to enhance binding affinity and selectivity towards specific protein targets.

Enhanced Scaffold Decoration and Morphing: The model supports advanced optimization tasks, such as scaffold decoration and morphing, which are crucial for modifying core structures to improve desired properties and transforming molecular frameworks to optimize biological activity and pharmacokinetics.

Overcomes Limitations of Traditional Representations: Traditional linear representations like SMILES and SELFIES are less effective in advanced optimization tasks. The SAFE representation considers structural fragments and their relationships, capturing the chemical and structural context more effectively.

Versatility in Masking Techniques: The method employs a variety of masking techniques, including random masking, substructure span masking, and motif reconstruction, which enhance the training of the encoder-decoder transformer model and improve the robustness of the generated compounds.

Integration with Variational Autoencoders (VAE): The integration of a VAE to generate a continuous latent space from the encoder-decoder transformer model allows for efficient compression and optimization of the latent space, resulting in the generation of diverse chemical compounds with specific properties.

Efficient Virtual Screening and Validation: The model includes steps for virtual screening and molecular dynamics methods to validate the generated compounds, ensuring that they meet the desired properties and binding affinities.

The disclosed techniques offer several applications including:

Drug Discovery and Development: The model can be used to generate novel chemical compounds with specific properties, accelerating the drug discovery process. It aids in designing compounds with enhanced binding affinity and selectivity towards protein targets, which is critical in developing effective drugs.

Medicinal Chemistry: The model supports scaffold decoration, linker design, and scaffold morphing, which are integral to medicinal chemistry for optimizing the pharmacokinetics and biological activity of compounds.

Protein-Ligand Interaction Studies: By generating protein graphical and textual embeddings and mapping them to the continuous latent space, the model can be applied in studies focused on understanding and optimizing protein-ligand interactions.

Chemical Compound Libraries: The method can be used to expand chemical libraries by generating a wide array of compounds with desired chemical and physical properties, which can be used in various chemical and biological research.

Personalized Medicine: The ability to tailor compounds to specific biological interactions and protein targets has potential applications in personalized medicine, where treatments can be customized based on individual genetic and protein profiles.

Agricultural Chemistry: The model can be applied to develop new agrochemicals with specific properties, such as improved efficacy and reduced environmental impact, by optimizing the chemical structures for desired biological interactions.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

It is to be understood that the above description is intended to be illustrative, and not restrictive. For example, the above-discussed embodiments may be used in combination with each other. Many other embodiments will be apparent to those of skill in the art upon reviewing the above description.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the embodiments.

While the present invention has been described with reference to particular embodiments, it should be understood that the embodiments are illustrative and that the scope of the invention is not limited to these embodiments. Many variations, modifications, additions, and improvements to the embodiments described above are possible. It is contemplated that these variations, modifications, additions, and improvements fall within the scope of the invention.

We claim:

1. A computer-implemented method for developing a generative chemistry model based on SAFE representations, the method comprising:
encoding one or more chemical compounds into the SAFE representations;
training an encoder-decoder transformer model based on the SAFE representations using one or more masking techniques, wherein the encoder-decoder transformer model creates a latent space to represent encoded SAFE representations of the chemical compounds;
training a variational Autoencoder (VAE) to generate a continuous latent space by compressing the latent space of the encoder-decoder transformer model; and
optimizing the continuous latent space to generate a plurality of chemical compounds with specific properties by decoding the SAFE representations of the chemical compounds.

2. The computer-implemented method of claim 1, wherein the one or more masking technique comprises random masking, substructure span masking, connecting node masking, linker design, random token corruption, substructure corruption, connecting node corruption, random span corruption, scaffold decoration, motif reconstruction, corrupt motif correction, motif extension and sequence to sequence fine tuning.

3. The computer-implemented method of claim 1, wherein the encoder-decoder transformer model is a Text-to-Text Transfer Transformer (T5) model.

4. The computer-implemented method of claim 1, wherein training the encoder-decoder transformer model comprising generating a plurality of hidden representations for the SAFE representations of chemical compounds.

5. The computer-implemented method of claim 1, wherein training the VAE comprising:
freezing model weights of the encoder-decoder transformer model; and
recreating the plurality of hidden representations from the continuous latent space.

6. The computer-implemented method of claim 1, wherein generating the continuous latent space comprising converting the latent space into a lower dimension continuous latent space for gradient based optimization.

7. The computer-implemented method of claim 1, wherein generating the plurality of chemical compounds with specific properties comprising:
generating a plurality of protein graphical and textual embeddings based on a protein-ligand dataset;
mapping the protein graphical and textual embeddings to decoding layers of the generative chemistry model;
decoding the SAFE representations of the chemical compounds based on the continuous latent space; and
validating the plurality of chemical compounds with specific properties using virtual screening and molecular dynamics methods.

8. The computer-implemented method of claim 7, wherein validating the plurality of chemical compounds with specific properties comprising:
masking binding motifs of the SAFE representations of the chemical compounds;
determining Tanimoto similarity between an unconditioned generation of chemical compounds and a protein conditioned generation of chemical compounds as ablation study; and
comparing with protein decoded SAFE representations and without protein decoded SAFE representations of the chemical compounds.

9. The computer-implemented method of claim 1, wherein the optimization of the continuous latent space is gradient-based optimization.

10. A computer-implemented system for developing a generative chemistry model based on SAFE representations, the computer-implemented system comprising: one or more computer processors, one or more computer readable memories, one or more computer readable storage devices, and program instructions stored on the one or more computer readable storage devices for execution by the one or more computer processors via the one or more computer readable memories, the program instructions comprising:
encoding one or more chemical compounds into the SAFE representations;
training an encoder-decoder transformer model based on the SAFE representations using one or more masking techniques, wherein the encoder-decoder transformer model creates a latent space to represent encoded SAFE representations of the chemical compounds;
training a variational Autoencoder (VAE) to generate a continuous latent space by compressing the latent space of the encoder-decoder transformer model; and
optimizing the continuous latent space to generate a plurality of chemical compounds with specific properties by decoding the SAFE representations of the chemical compounds.

11. The computer-implemented system of claim 10, wherein the one or more masking technique comprises random masking, substructure span masking, connecting node masking, linker design, random token corruption, substructure corruption, connecting node corruption, random span corruption, scaffold decoration, motif reconstruction, corrupt motif correction, motif extension and sequence to sequence fine tuning.

12. The computer-implemented system of claim 10, wherein the encoder-decoder transformer model is a Text-to-Text Transfer Transformer (T5) model.

13. The computer-implemented system of claim 10, wherein training the encoder-decoder transformer model comprising generating a plurality of hidden representations for the SAFE representations of chemical compounds.

14. The computer-implemented system of claim 10, wherein training the VAE comprising:
freezing model weights of the encoder-decoder transformer model; and
recreating the plurality of hidden representations from the continuous latent space.

15. The computer-implemented system of claim 10, wherein generating the continuous latent space comprising converting the latent space into a lower dimension continuous latent space for gradient based optimization.

16. The computer-implemented system of claim 10, wherein generating the plurality of chemical compounds with specific properties comprising:
generating a plurality of protein graphical and textual embeddings based on a protein-ligand dataset;
mapping the protein graphical and textual embeddings to decoding layers of the generative chemistry model;
decoding the SAFE representations of the chemical compounds based on the continuous latent space; and
validating the plurality of chemical compounds with specific properties using virtual screening and molecular dynamics methods.

17. The computer-implemented system of claim 16, wherein validating the plurality of chemical compounds with specific properties comprising:
masking binding motifs of the SAFE representations of the chemical compounds;
determining Tanimoto similarity between an unconditioned generation of chemical compounds and a protein conditioned generation of chemical compounds as ablation study; and
comparing with protein decoded SAFE representations and without protein decoded SAFE representations of the chemical compounds.

18. The computer-implemented system of claim 10, wherein the optimization of the continuous latent space is gradient-based optimization.

19. A non-transitory computer-readable storage medium having stored thereon computer executable instruction which when executed by one or more processors, cause the one or more processors to carry out operations for developing a generative chemistry model based on SAFE representations, the operations comprising perform the operations comprising:
encoding one or more chemical compounds into the SAFE representations;
training an encoder-decoder transformer model based on the SAFE representations using one or more masking techniques, wherein the encoder-decoder transformer model creates a latent space to represent encoded SAFE representations of the chemical compounds;
training a variational Autoencoder (VAE) to generate a continuous latent space by compressing the latent space of the encoder-decoder transformer model; and
optimizing the continuous latent space to generate a plurality of chemical compounds with specific properties by decoding the SAFE representations of the chemical compounds.

20. The computer-implemented method of claim 19, wherein the encoder-decoder transformer model is a Text-to-Text Transfer Transformer (T5) model.
